(19) United States
(12) Patent Application Publication
(10) Pub. No.: US 2026/0242805 A1
(43) Pub. Date: Aug. 20, 2026

(54) CIRCULAR RNA VECTORS COMPRISING MODIFIED NUCLEOTIDES

(71) Applicant: UNIVERSITY OF MASSACHUSETTS, Westborough, MA (US)

(72) Inventors: Li LI, Westborough, MA (US); Alejandro FELIX MEJIA, Worcester, MA (US)

(21) Appl. No.: 19/637,632

(22) Filed: Apr. 2, 2026

Related U.S. Application Data
(63) Continuation of application No. PCT/US2024/049526, filed on Oct. 2, 2024.
(60) Provisional application No. 63/587,270, filed on Oct. 2, 2023.

(51) Int. Cl. C12N 15/68

(52) U.S. Cl. CPC C12N 15/68; C12N 2310/335; C12N 2310/532; C12N 2800/60; C12N 2830/42; C12N 2840/203

(57) ABSTRACT

Provided is an RNA vector comprising: a permuted group 1 intron/exon comprising a 5′ exon, a 3′ exon, and an intron, the intron comprising a 5′ intron end and a 3′ intron end; and at least one modified nucleotide; wherein the 3′ exon is upstream of the 5′ exon; wherein a portion of the intron including the 3′ intron end is upstream of the 3′ exon; wherein a portion of the intron including the 5′ intron end is downstream of the 5′ exon; and wherein the permuted group 1 intron/exon is active in the presence of the modified nucleotide. Also provided is a DNA vector and methods for expressing a target protein from a circular RNA vector.

### CROSS REFERENCE TO RELATED APPLICATIONS

This application claims the benefit of priority to U.S. Provisional Application No. 63/587,270, filed Oct. 2, 2023, which is incorporated by reference herein in its entirety.

### REFERENCE TO SEQUENCE LISTING

The sequence listing submitted on Oct. 2, 2024, as an .XML file entitled “11579-011WO1_ST26.xml” created on Sep. 26, 2024, and having a file size of 39,572 bytes is hereby incorporated by reference pursuant to 37 C.F.R. § 1.52(e)(5).

### BACKGROUND

Linear mRNAs containing modified nucleosides (such as those containing pseudouridine and N1-methylpseudouridine) can be prepared by in vitro transcription. These nucleoside-modified linear mRNAs are important gene therapy vectors with reduced immunogenicity. There is a need to incorporate such modified nucleosides into circular mRNAs, which have a longer half-life than linear mRNAs.

Circular mRNAs contain an internal ribosome entry site (450-750 nucleotides) necessary for translation and an open reading frame that encodes the therapeutic protein. Therefore, to express any therapeutic protein longer than 200 amino acids, a circular mRNA containing more than 1,000 nucleotides is needed. However, there is no available method to incorporate these modified nucleosides into circular mRNAs with a length of more than 1,000 nucleotides.

### SUMMARY

Disclosed herein are circular mRNA platforms for expressing target proteins. Further disclosed herein are circular mRNA platforms for transient gene therapy with reduced immunogenicity and reactogenicity which can be translated in the presence of modified nucleotides (for example, pseudouridine and N1-methylpseudouridine). Also disclosed herein are the methods and constructs for preparing these nucleoside-modified circular mRNAs.

In one aspect, provided is an RNA vector comprising: a permuted group 1 intron/exon comprising a 5′ exon, a 3′ exon, and an intron, the intron comprising a 5′ intron end and a 3′ intron end; and at least one modified nucleotide; wherein the 3′ exon is upstream of the 5′ exon; wherein a portion of the intron including the 3′ intron end is upstream of the 3′ exon; wherein a portion of the intron including the 5′ intron end is downstream of the 5′ exon; and wherein the permuted group 1 intron/exon is active in the presence of the modified nucleotide.

In another aspect, provided is a circular RNA vector formed from any of the disclosed RNA vectors.

In yet another aspect, provided is a DNA vector comprising: a permuted group 1 intron/exon comprising a 5′ exon, a 3′ exon, and an intron, the intron comprising a 5′ intron end and a 3′ intron end; and multiple cloning cite; wherein the 3′ exon is upstream of the 5′ exon; wherein a portion of the intron including the 3′ intron end is upstream of the 3′ exon; and wherein a portion of the intron including the 5′ intron end is downstream of the 5′ exon.

In yet still another aspect, provided is a circular RNA vector transcribed from any of the disclosed DNA vectors.

In yet still another aspect, provided is a method for expressing a target protein from a circular RNA vector comprising: providing any of the disclosed circular RNA vectors; and translating the target protein.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIGS. 1A-1B show that RNA can be circularized without proteins.

FIG. 2 depicts methods used for investigating how the m1Ψ modification prevents permuted group 1 intron circularization of synthetic mRNAs.

FIG. 3 depicts different uridine analogs used to generate fully uridine-modified mRNAs.

FIGS. 4A-4B demonstrate that both the linear Azoarcus (FIG. 4A) and PIE Azoarcus (FIG. 4B) constructs were prepared by IVT using different ratios of U to m1Ψ. FIG. 4C shows the quantification of the amount of precursor remaining for both FIG. 4A and FIG. 4B.

FIG. 5A shows a diagram of directed evolution of fully-uridine substituted group 1 introns. FIG. 5B shows the number of mutations per molecules from the prepared library.

FIG. 5C shows the mutation types present in the mutant library. FIG. 5D shows the activity of the mutant library compared to the wildtype sequence.

FIG. 6 shows the mechanism of Group I intron-PIE splicing.

FIG. 7 depicts that self vs. non-self-recognition protects host from invading pathogens.

FIG. 8 shows a schematic of the experimental workflow used to investigate TLR7/8 sensing of exogenous circRNAs.

FIGS. 9A-9C show circRNA preparation.

FIGS. 10A-10B show circRNA immunogenicity.

FIGS. 11A-11C show verification of CRISPR KO of gene-of-interest in transdifferentiated BLaER1 cells.

FIGS. 12A-12B depict that circRNA sensing is TLR7/8-mediated.

FIGS. 13A-13E depict how ψ/m1ψ modification reduces group I intron activity by weakening the interaction between the GAAA tetraloop and its receptor.

FIG. 14 depicts the effects of pseudouridine and N1-methylpseudouridine on splicing efficiency of the Anabaena PIE.

FIGS. 15A-15B depict the effects of pseudouridine and N1-methylpseudouridine on splicing efficiency of the Anabaena PIE and precursor remaining over time.

FIG. 16 depicts the effects of pseudouridine and N1-methylpseudouridine on splicing efficiency of the Azoarcus PIE.

FIGS. 17A-17B depict the effects of pseudouridine and N1-methylpseudouridine on splicing efficiency of the Azoarcus PIE and precursor remaining over time.

FIGS. 18A-18B depict that the Azoarcus PIE P6 is circularized efficiently and can successfully make modified circular mRNA. FIG. 18C depicts that the circular mRNA does not accumulate.

FIG. 19 depicts the circularization efficiency of Azoarcus PIE P8.

FIG. 20 shows the results of a 6% denaturing PAGE, SYBR gold stained assay which demonstrates the circularization of Ψ-modified RNA with greater than 90% efficiency.

FIG. 21 depicts that the improved Azoarcus PIE generates a precise exon-exon junction.

FIG. 22 is a schematic of the permuted intron-exon (PIE) strategy for circularization.

FIGS. 23A-23B depict 15% circularization efficiency for a 1.5 kb pseudouridine-modified mRNA.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

In one aspect, provided is an RNA vector including: a permuted group 1 intron/exon comprising a 5′ exon, a 3′ exon, and an intron, the intron comprising a 5′ intron end and a 3′ intron end; and at least one modified nucleotide; wherein the 3′ exon is upstream of the 5′ exon; wherein a portion of the intron including the 3′ intron end is upstream of the 3′ exon; wherein a portion of the intron including the 5′ intron end is downstream of the 5′ exon; and wherein the permuted group 1 intron/exon is active in the presence of the modified nucleotide. In some aspects, the RNA vector can further include a nucleic acid sequence encoding a target protein.

In another aspect, provided is an RNA vector including: a permuted group 1 intron/exon comprising a 5′ exon, a 3′ exon, and an intron, the intron comprising a 5′ intron end and a 3′ intron end; a nucleic acid sequence encoding a target protein; and at least one modified nucleotide; wherein the 3′ exon is upstream of the 5′ exon; wherein a portion of the intron including the 3′ intron end is upstream of the 3′ exon; wherein a portion of the intron including the 5′ intron end is downstream of the 5′ exon; and wherein the permuted group 1 intron/exon is active in the presence of the modified nucleotide. In some aspects, the nucleic acid sequence encoding the target protein can be between the 3′ exon and the 5′ exon.

In some aspects, the modified nucleotide can be a modified uridine nucleotide, for example, pseudouridine, N1-methylpseudouridine, 5-methyluridine, 5-methoxyuridine, 5-methylcytidine, 1-ethylpseudouridine, 2-thiouridine, 4-thiouridine, 2-thio-1-methyl-pseudouridine, 2-thio-5-aza-uridine, 4-thio-1-methyl-pseudouridine, 5-thio-pseudouridine, 5-aza-uridine, or any combination thereof. In some such aspects, some or all uridine nucleotides in the RNA vector may be replaced with modified uridine nucleotides, or any combination thereof. For example, in some aspects, greater than about 0% (e.g., greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, about 100%) of the uridine nucleotides in the RNA vector may be replaced with modified uridine nucleotides. In some aspects, up to about 100% (e.g., up to about 95%, up to about 90%, up to about 85%, up to about 80%, up to about 75%, up to about 70%, up to about 65%, up to about 60%, up to about 55%, up to about 50%, up to about 45%, up to about 40%, up to about 35%, up to about 30%, up to about 25%, up to about 20%, up to about 15%, up to about 10%, up to about 5%) of the uridine nucleotides in the RNA vector may be replaced with modified uridine nucleotides.

It is considered that any percentage of the uridine nucleotides in the RNA vector may be replaced with modified uridine nucleotides. For example, in some aspects, from about 0% to about 100% (e.g., from about 5% to about 95%, from about 10% to about 90%, from about 15% to about 85%, from about 20% to about 80%, from about 25% to about 75%, from about 30% to about 70%, from about 35% to about 65%, from about 40% to about 60%, from about 45% to about 55%, from about 0% to about 50%, from about 5% to about 45%, from about 10% to about 40%, from about 15% to about 35%, from about 20% to about 30%, from about 50% to about 100%, from about 55% to about 95%, from about 60% to about 90%, from about 65% to about 85%, from about 70% to about 80%) of the uridine nucleotides in the RNA vector may be replaced with modified uridine nucleotides.

Additionally or alternatively, in other aspects, the modified nucleotide can be a modified adenosine nucleotide, a modified cytidine nucleotide, a modified guanosine nucleotide, or any combination thereof.

It is understood that the at least one modified nucleotide may be present in any portion of the RNA vector. For example, in some aspects, the at least one modified nucleotide may be present in the permuted group 1 intron/exon. In other aspects, the at least one modified nucleotide may be present in the nucleic acid sequence encoding a target protein. In yet other aspects, both the permuted group 1 intron/exon and the nucleic acid sequence encoding a target protein may each include at least one modified nucleotide.

In some aspects, the permuted group 1 intron/exon can be an Azoarcus group I intron/exon. In some aspects, the permuted group 1 intron/exon can further include at least one mutation to a wildtype group 1 intron/exon. In some aspects, the wildtype group 1 intron/exon can be an Azoarcus group 1 intron/exon. In some aspects, the mutation can improve transcription of the vector. In some aspects, the mutation can include addition and/or removal of nucleotides to/from the wildtype group 1 intron/exon.

In some aspects, the portion of the intron including the 3′ intron end can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 15 or SEQ ID NO: 16. In some aspects, the portion of the intron including the 3′ intron end can include SEQ ID NO: 15 or SEQ ID NO: 16.

In some aspects, the portion of the intron including the 5′ intron end can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 17 or SEQ ID NO: 18. In some aspects, the portion of the intron including the 5′ intron end can include SEQ ID NO: 17 or SEQ ID NO: 18.

In some aspects, the 3′ exon can include 80% similarity or more to (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) SEQ ID NO: 19 or SEQ ID NO: 20. In some aspects the 3′ exon can include SEQ ID NO: 19 or SEQ ID NO: 20.

In some aspects, the 5′ exon can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 21 or SEQ ID NO: 22. In some aspects, the 5′ exon can include SEQ ID NO: 21 or SEQ ID NO: 22.

In some aspects, the target protein can be any protein of interest. For example, in some aspects, the target protein can be an antigen (e.g., COVID spike protein), a hormone (e.g., insulin, erythropoietin, human growth hormone), a cytokine (e.g., an interferon), or a clotting factor (e.g., factor IX). Additionally or alternatively, in other aspects, the target protein can include a fluorescent protein. In some such aspects, the target protein can include eGFP, mCherry, Firefly luciferase, or NanoLuc luciferase. An example RNA vector encoding eGFP is provided in SEQ ID NO: 26.

In some aspects, the target protein can include at least one modified nucleotide. In some such aspects, the permuted group 1 intron/exon may additionally include at least one modified nucleotide. In other such aspects, the permuted group 1 intron/exon may not include any modified nucleotides. In some aspects, the modified nucleotide can be a modified uridine nucleotide, for example, pseudouridine, N1-methylpseudouridine, 5-methyluridine, 5-methoxyuridine, 5-methylcytidine, 1-ethylpseudouridine, 2-thiouridine, 4-thiouridine, 2-thio-1-methyl-pseudouridine, 2-thio-5-aza-uridine, 4-thio-1-methyl-pseudouridine, 5-thio-pseudouridine, 5-aza-uridine, or any combination thereof. In some such aspects, some or all uridine nucleotides in the nucleic acid sequence encoding the target protein may be replaced with modified uridine nucleotides. For example, in some aspects, greater than about 0% (e.g., greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, about 100%) of the uridine nucleotides in the nucleic acid sequence encoding the target protein may be replaced with modified uridine nucleotides. In some aspects, up to about 100% (e.g., up to about 95%, up to about 90%, up to about 85%, up to about 80%, up to about 75%, up to about 70%, up to about 65%, up to about 60%, up to about 55%, up to about 50%, up to about 45%, up to about 40%, up to about 35%, up to about 30%, up to about 25%, up to about 20%, up to about 15%, up to about 10%, up to about 5%) of the uridine nucleotides in the nucleic acid sequence encoding the target protein may be replaced with modified uridine nucleotides.

It is considered that any percentage of the uridine nucleotides in the nucleic acid sequence encoding the target protein may be replaced with modified uridine nucleotides. For example, in some aspects, from about 0% to about 100% (e.g., from about 5% to about 95%, from about 10% to about 90%, from about 15% to about 85%, from about 20% to about 80%, from about 25% to about 75%, from about 30% to about 70%, from about 35% to about 65%, from about 40% to about 60%, from about 45% to about 55%, from about 0% to about 50%, from about 5% to about 45%, from about 10% to about 40%, from about 15% to about 35%, from about 20% to about 30%, from about 50% to about 100%, from about 55% to about 95%, from about 60% to about 90%, from about 65% to about 85%, from about 70% to about 80%) of the uridine nucleotides in the nucleic acid sequence encoding the target protein may be replaced with modified uridine nucleotides.

In some aspects, the RNA vector can further include an internal ribosome entry site (IRES). In some aspects, the IRES can be active in the presence of the modified nucleotide. In some aspects, the IRES can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 23. In some aspects, the IRES can include SEQ ID NO: 23.

In some aspects, the RNA vector can further include at least one spacer. In some aspects, the spacer can be selected at each occurrence from a sequence including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 10 or SEQ ID NO: 11. In some aspects, the spacer can be selected at each occurrence from SEQ ID NO: 10 or SEQ ID NO: 11.

In some aspects, the RNA vector can be greater than about 1000 nucleotides (e.g., greater than about 1100 nucleotides, greater than about 1200 nucleotides, greater than about 1300 nucleotides, greater than about 1400 nucleotides, greater than about 1500 nucleotides, greater than about 1600 nucleotides, greater than about 1700 nucleotides, greater than about 1800 nucleotides, greater than about 2000 nucleotides, greater than about 2200 nucleotides, greater than about 2400 nucleotides, greater than about 2600 nucleotides, greater than about 2800 nucleotides, greater than about 3000 nucleotides, greater than about 3200 nucleotides, greater than about 3400 nucleotides, greater than about 3600 nucleotides, greater than about 3800 nucleotides, greater than about 4000 nucleotides, greater than about 4200 nucleotides, greater than about 4400 nucleotides, greater than about 4600 nucleotides, greater than about 4800 nucleotides, greater than about 5000 nucleotides) in length.

In some aspects, the RNA vector can be circularized.

In yet another aspect, provided is a method of preparing a circular RNA vector, the method comprising circularizing any of the disclosed RNA vectors. In some aspects, the 5′ intron end can at least partially hybridize with the 3′ intron end, thereby forming the circular RNA vector. In some aspects, the method can further include splicing the intron.

In some aspects, the RNA vector can be circularized by in vitro transcription (IVT). In some aspects, IVT can be carried out at a temperature of at least about 30° C. (e.g., at least about 32° C., at least about 34° C., at least about 36° C., at least about 38° C., at least about 40° C., at least about 42° C., at least about 44° C., at least about 46° C., at least about 48° C., at least about 50° C., at least about 52° C., at least about 54° C., at least about 56° C., at least about 58° C., at least about 60° C., at least about 62° C., at least about 64° C., at least about 66° C., at least about 68° C., at least about 70° C.). In some aspects, IVT can be carried out at a temperature of up to about 70° C. (e.g., up to about 68° C., up to about 66° C., up to about 64° C., up to about 62° C., up to about 60° C., up to about 58° C., up to about 56° C., up to about 54° C., up to about 52° C., up to about 50° C., up to about 48° C., up to about 46° C., up to about 44° C., up to about 42° C., up to about 40° C., up to about 38° C., up to about 36° C., up to about 34° C., up to about 32° C., up to about 30° C.).

It is considered that IVT can be carried out at a temperature ranging from any of the minimum values described above to any of the maximum values described above. For example, in some aspects, IVT can be carried out at a temperature of from about 30° C. to about 70° C. (e.g., from about 32° C. to about 68° C., from about 34° C. to about 66° C., from about 36° C. to about 64° C., from about 38° C. to about 62° C., from about 40° C. to about 60° C., from about 42° C. to about 58° C., from about 44° C. to about 56° C., from about 46° C. to about 54° C., from about 48° C. to about 52° C., from about 30° C. to about 50° C., from about 32° C. to about 48° C., from about 34° C. to about 46° C., from about 36° C. to about 44° C., from about 38° C. to about 42° C., from about 50° C. to about 70° C., from about 52° C. to about 68° C., from about 54° C. to about 66° C., from about 56° C. to about 64° C., from about 58° C. to about 62° C.).

In yet still another aspect, provided is a circular RNA vector formed from any of the disclosed RNA vectors and including at least one modified nucleotide. In some aspects, the modified nucleotide can be a modified uridine nucleotide, for example, pseudouridine, N1-methylpseudouridine, 5-methyluridine, 5-methoxyuridine, 5-methylcytidine, 1-ethylpseudouridine, 2-thiouridine, 4-thiouridine, 2-thio-1-methyl-pseudouridine, 2-thio-5-aza-uridine, 4-thio-1-methyl-pseudouridine, 5-thio-pseudouridine, 5-aza-uridine, or any combination thereof. In some such aspects, some or all uridine nucleotides in the circular RNA vector may be replaced with modified uridine nucleotides. For example, in some aspects, greater than about 0% (e.g., greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, about 100%) of the uridine nucleotides in the nucleic acid sequence encoding the target protein may be replaced with modified uridine nucleotides. In some aspects, up to about 100% (e.g., up to about 95%, up to about 90%, up to about 85%, up to about 80%, up to about 75%, up to about 70%, up to about 65%, up to about 60%, up to about 55%, up to about 50%, up to about 45%, up to about 40%, up to about 35%, up to about 30%, up to about 25%, up to about 20%, up to about 15%, up to about 10%, up to about 5%) of the uridine nucleotides in the circular RNA vector may be replaced with modified uridine nucleotides.

It is considered that any percentage of the uridine nucleotides in the circular RNA vector may be replaced with modified uridine nucleotides. For example, in some aspects, from about 0% to about 100% (e.g., from about 5% to about 95%, from about 10% to about 90%, from about 15% to about 85%, from about 20% to about 80%, from about 25% to about 75%, from about 30% to about 70%, from about 35% to about 65%, from about 40% to about 60%, from about 45% to about 55%, from about 0% to about 50%, from about 5% to about 45%, from about 10% to about 40%, from about 15% to about 35%, from about 20% to about 30%, from about 50% to about 100%, from about 55% to about 95%, from about 60% to about 90%, from about 65% to about 85%, from about 70% to about 80%) of the uridine nucleotides in the circular RNA vector may be replaced with modified uridine nucleotides.

In yet still another aspect, provided is a DNA vector comprising: a permuted group 1 intron/exon comprising a 5′ exon, a 3′ exon, and an intron, the intron comprising a 5′ intron end and a 3′ intron end; wherein the 3′ exon is upstream of the 5′ exon; wherein a portion of the intron including the 3′ intron end is upstream of the 3′ exon; and wherein a portion of the intron including the 5′ intron end is downstream of the 5′ exon. In some aspects, the DNA vector can further include a multiple cloning site. Additionally or alternatively, in other aspects, the DNA vector can further include a nucleic acid sequence encoding a target protein. Additionally or alternatively, in yet other aspects, the DNA vector can further include an internal ribosome entry site (IRES).

In yet still another aspect, provided is an DNA vector including: a permuted group 1 intron/exon comprising a 5′ exon, a 3′ exon, and an intron, the intron comprising a 5′ intron end and a 3′ intron end; and a multiple cloning site; wherein the 3′ exon is upstream of the 5′ exon; wherein a portion of the intron including the 3′ intron end is upstream of the 3′ exon; wherein a portion of the intron including the 5′ intron end is downstream of the 5′ exon; and wherein the permuted group 1 intron/exon is active in the presence of the modified nucleotide. In some aspects, the multiple cloning site can be between the 3′ exon and the 5′ exon.

In yet still another aspect, provided is an DNA vector including: a permuted group 1 intron/exon comprising a 5′ exon, a 3′ exon, and an intron, the intron comprising a 5′ intron end and a 3′ intron end; and a nucleic acid sequence encoding a target protein; wherein the 3′ exon is upstream of the 5′ exon; wherein a portion of the intron including the 3′ intron end is upstream of the 3′ exon; wherein a portion of the intron including the 5′ intron end is downstream of the 5′ exon; and wherein the permuted group 1 intron/exon is active in the presence of the modified nucleotide. In some aspects, the nucleic acid sequence encoding the target protein can be between the 3′ exon and the 5′ exon. In some aspects, the DNA vector can further include an internal ribosome entry site (IRES).

In some aspects, the permuted group 1 intron/exon can be an Azoarcus group I intron/exon. In some aspects, the permuted group 1 intron/exon can further include at least one mutation to a wildtype group 1 intron/exon. In some aspects, the wildtype group 1 intron/exon can be an Azoarcus group 1 intron/exon and can include SEQ ID NO: 1. In some aspects, the mutation can improve transcription of the vector. In some aspects, the mutation can include addition and/or removal of nucleotides to/from the wildtype group 1 intron/exon.

In some aspects, the permuted group 1 intron/exon can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 1. In some aspects, the permuted group 1 intron/exon can include SEQ ID NO: 1.

In some aspects, the portion of the intron including the 3′ intron end can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 2 or SEQ ID NO: 3. In some aspects, the portion of the intron including the 3′ intron end can include SEQ ID NO: 2 or SEQ ID NO: 3.

In some aspects, the portion of the intron including the 5′ intron end can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 4 or SEQ ID NO: 5. In some aspects, the portion of the intron including the 5′ intron end can include SEQ ID NO: 4 or SEQ ID NO: 5.

In some aspects, the 5′ exon can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 6. In some aspects, the 5′ exon can include SEQ ID NO: 6.

In some aspects, the 3′ exon can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 7. In some aspects, the 3′ exon can include SEQ ID NO: 7.

In some aspects, the multiple cloning site can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 8 or SEQ ID NO: 9. In some aspects, the multiple cloning site can include SEQ ID NO: 8 or SEQ ID NO: 9.

In some aspects, the target protein can be any protein of interest. For example, in some aspects, the target protein can be an antigen (e.g., COVID spike protein), a hormone (e.g., insulin, erythropoietin, human growth hormone), a cytokine (e.g., an interferon), or a clotting factor (e.g., factor IX). Additionally or alternatively, in other aspects, the target protein can include a fluorescent protein. In some such aspects, the target protein can include eGFP, mCherry, Firefly luciferase, or NanoLuc luciferase.

In some aspects, the DNA vector can further include at least one spacer. In some aspects, the spacer can be selected at each occurrence from a sequence having 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 10 or SEQ ID NO: 11. In some aspects, the spacer can be selected at each occurrence from SEQ ID NO: 10 or SEQ ID NO: 11.

In some aspects, the DNA vector can be greater than about 1000 nucleotides (e.g., greater than about 1100 nucleotides, greater than about 1200 nucleotides, greater than about 1300 nucleotides, greater than about 1400 nucleotides, greater than about 1500 nucleotides, greater than about 1600 nucleotides, greater than about 1700 nucleotides, greater than about 1800 nucleotides, greater than about 2000 nucleotides, greater than about 2200 nucleotides, greater than about 2400 nucleotides, greater than about 2600 nucleotides, greater than about 2800 nucleotides, greater than about 3000 nucleotides, greater than about 3200 nucleotides, greater than about 3400 nucleotides, greater than about 3600 nucleotides, greater than about 3800 nucleotides, greater than about 4000 nucleotides, greater than about 4200 nucleotides, greater than about 4400 nucleotides, greater than about 4600 nucleotides, greater than about 4800 nucleotides, greater than about 5000 nucleotides) in length.

In some aspects, the DNA vector can be circularized.

In yet another aspect, provided is a method of preparing a circular RNA vector, the method comprising: a) transcribing any of the disclosed DNA vectors into an RNA vector; and b) circularizing the RNA vector. In some aspects, the method can further include: c) modifying at least one nucleotide in the circular RNA vector. In some aspects, the DNA vector can be transcribed in vitro, in vivo, or ex vivo. In some aspects, the RNA vector can be any of the disclosed RNA vectors.

In some aspects, the 5′ intron end can at least partially hybridize with the 3′ intron end, thereby forming the circular RNA vector. In some aspects, the method can further include splicing the intron.

In some aspects, the RNA vector can be circularized by in vitro transcription (IVT). In some aspects, IVT can be carried out at a temperature of at least about 30° C. (e.g., at least about 32° C., at least about 34° C., at least about 36° C., at least about 38° C., at least about 40° C., at least about 42° C., at least about 44° C., at least about 46° C., at least about 48° C., at least about 50° C., at least about 52° C., at least about 54° C., at least about 56° C., at least about 58° C., at least about 60° C., at least about 62° C., at least about 64° C., at least about 66° C., at least about 68° C., at least about 70° C.). In some aspects, IVT can be carried out at a temperature of up to about 70° C. (e.g., up to about 68° C., up to about 66° C., up to about 64° C., up to about 62° C., up to about 60° C., up to about 58° C., up to about 56° C., up to about 54° C., up to about 52° C., up to about 50° C., up to about 48° C., up to about 46° C., up to about 44° C., up to about 42° C., up to about 40° C., up to about 38° C., up to about 36° C., up to about 34° C., up to about 32° C., up to about 30° C.).

It is considered that IVT can be carried out at a temperature ranging from any of the minimum values described above to any of the maximum values described above. For example, in some aspects, IVT can be carried out at a temperature of from about 30° C. to about 70° C. (e.g., from about 32° C. to about 68° C., from about 34° C. to about 66° C., from about 36° C. to about 64° C., from about 38° C. to about 62° C., from about 40° C. to about 60° C., from about 42° C. to about 58° C., from about 44° C. to about 56° C., from about 46° C. to about 54° C., from about 48° C. to about 52° C., from about 30° C. to about 50° C., from about 32° C. to about 48° C., from about 34° C. to about 46° C., from about 36° C. to about 44° C., from about 38° C. to about 42° C., from about 50° C. to about 70° C., from about 52° C. to about 68° C., from about 54° C. to about 66° C., from about 56° C. to about 64° C., from about 58° C. to about 62° C.).

In yet still another aspect, provided is a circular RNA vector formed from any of the disclosed DNA vectors and including at least one modified nucleotide. In some aspects, the modified nucleotide can be a modified uridine nucleotide, for example, pseudouridine, N1-methylpseudouridine, 5-methyluridine, 5-methoxyuridine, 5-methylcytidine, 1-ethylpseudouridine, 2-thiouridine, 4-thiouridine, 2-thio-1-methyl-pseudouridine, 2-thio-5-aza-uridine, 4-thio-1-methyl-pseudouridine, 5-thio-pseudouridine, 5-aza-uridine, or any combination thereof. In some such aspects, some or all uridine nucleotides in the circular RNA vector may be replaced with modified uridine nucleotides. For example, in some aspects, greater than about 0% (e.g., greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, about 100%) of the uridine nucleotides in the nucleic acid sequence encoding the target protein may be replaced with modified uridine nucleotides. In some aspects, up to about 100% (e.g., up to about 95%, up to about 90%, up to about 85%, up to about 80%, up to about 75%, up to about 70%, up to about 65%, up to about 60%, up to about 55%, up to about 50%, up to about 45%, up to about 40%, up to about 35%, up to about 30%, up to about 25%, up to about 20%, up to about 15%, up to about 10%, up to about 5%) of the uridine nucleotides in the circular RNA vector may be replaced with modified uridine nucleotides.

It is considered that any percentage of the uridine nucleotides in the circular RNA vector may be replaced with modified uridine nucleotides. For example, in some aspects, from about 0% to about 100% (e.g., from about 5% to about 95%, from about 10% to about 90%, from about 15% to about 85%, from about 20% to about 80%, from about 25% to about 75%, from about 30% to about 70%, from about 35% to about 65%, from about 40% to about 60%, from about 45% to about 55%, from about 0% to about 50%, from about 5% to about 45%, from about 10% to about 40%, from about 15% to about 35%, from about 20% to about 30%, from about 50% to about 100%, from about 55% to about 95%, from about 60% to about 90%, from about 65% to about 85%, from about 70% to about 80%) of the uridine nucleotides in the circular RNA vector may be replaced with modified uridine nucleotides.

While a number of sequences herein are disclosed as DNA sequences, the corresponding RNA sequences are also disclosed herein (i.e., where the thymines are replaced by uracils).

In yet still another aspect, provided is a method for expressing a target protein from a circular RNA vector including: providing any of the disclosed circular RNA vectors; and translating the target protein.

### EXAMPLES

### Example 1:1-Methylpseudouridine Inhibits the Group 1 Intron Facilitated Circularization of Synthetic mRNAs

The effectiveness of mRNA-based therapeutics can be significantly improved by combining RNA modification and RNA circularization to further increase the performance of synthetic mRNAs and lead to more effective therapeutics. Compared to linear mRNAs, they are more durable because they are resistant to exonuclease degradation. However, the most effective mRNA circularization strategy, one based on the permuted group I intron, is currently incompatible with widely used modified nucleotides such as 1-methylpseudouridine (m1° C.) 1. This hampers the development of fully modified circular mRNAs as a new mRNA therapeutic platform. Because these modified nucleotides are crucial in reducing the immunostimulatory effects and the reactogenicity of mRNA therapeutics (PMID: 16111635), a fully modified circular mRNA platform is expected to significantly extend the duration of mRNA therapeutics while having a reactogenicity profile similar to the current FDA-approved linear modified mRNAs.

A study was conducted which investigated how the m1 modification prevents permuted group 1 intron circularization of synthetic mRNAs and how to potentially overcome this challenge using directed evolution.

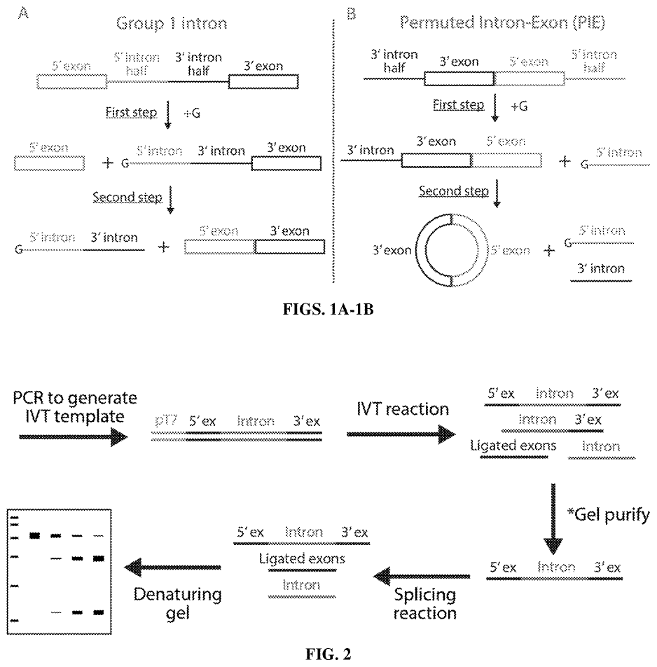

RNA can be circularized without proteins as shown in FIGS. 1A-1B which are diagrams showing the group 1 intron (FIG. 1A) and permuted intron-exon reactions (FIG. 1B). For the PIE construct, the group 1 intron sequence is split in halves and rearranged so that the intron halves surround their exons.

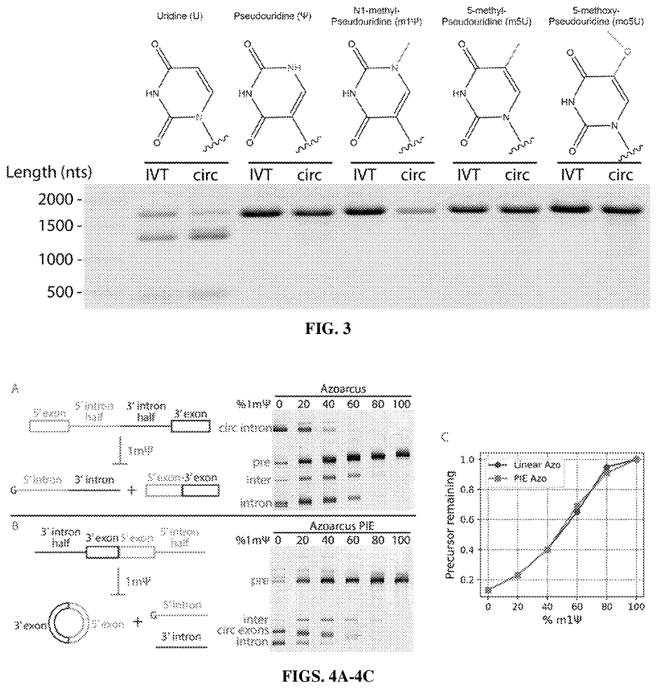

The methods used in this study are shown in FIG. 2. In vitro transcription (IVT) templates were prepared by PCR amplification from a plasmid containing the sequence of interest. IVT was done at 37° C. for 2 hours, substituting UTP for Uridine analogs depending on the reaction. For time course experiments, gel purification of the IVT RNA was performed so that only precursor RNA was present at t=0. For RNAs>500 nts, agarose gel were used. For RNA<500 nts, polyacrylamide gels were used.

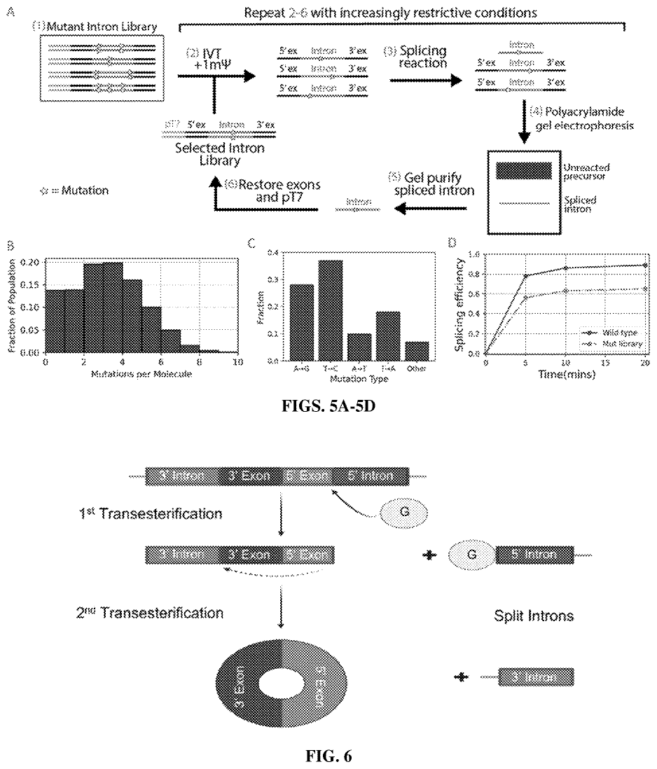

FIG. 3, FIGS. 4A-4C, and FIGS. 5A-5D show the results of this study. The top portion of FIG. 3 shows the different uridine analogs used to generate fully uridine-modified mRNAs. An mRNA containing the EMCV IRES and eGFP was prepared through IVT using different uridine analogs. The mRNA was cleaned up by precipitation followed by the circularization reaction. 500 ng of RNA was treated with glyoxal to denature and visualized by agarose electrophoresis. FIGS. 4A-4C demonstrate that both the linear Azoarcus (FIG. 4A) and PIE Azoarcus (FIG. 4B) constructs were prepared by IVT using different ratios of U to m1Ψ. The resulting RNAs were cleaned up using spin columns. The RNAs were spliced/circularized at 40° C. for 10 minutes. RNA was visualized using PAGE. FIG. 4C shows the quantification of the amount of precursor remaining for both FIG. 4A and FIG. 4B.

FIG. 5A shows a diagram of directed evolution of fully-uridine substituted group 1 introns. A library of mutant group 1 intron variants is prepared through error-prone PCR. This is followed by IVT, splicing reaction, and a PAGE-based selection based on the size of the spliced introns. Finally, spliced introns are recovered via gel extraction and turned into templates for IVT through RT and PCR. Finally, the restored templates are used for the next round of selection. FIG. 5B shows the number of mutations per molecules from the prepared library. FIG. 5C shows the mutation types present in the mutant library. FIG. 5D shows the activity of the mutant library compared to the wildtype sequence.

This study determined that the Azoarcus group 1 intron can be used to generate circular RNA using the PIE method. The linear form of the Azoarcus group 1 intron has a similar pattern of inhibition by m1l′ as the PIE construct and can be used to study the inactivating effects of uridine analogs. The intron mutant library contains mutations that deplete Uridine residues. In future studies, chemical probing can be used to determine secondary structural changes resulting from uridine modification. Directed evolution can also be used to find group 1 intron variants that are active when modified

### Example 2: TLR7/8 Sensing of Exogenous Circrnas Activates an Innate Immune Response in Human Cells

Circular RNA (circRNA) is a ubiquitously expressed class of RNA that forms a unique covalently closed-loop structure. Due to its circular topology, circRNAs are more stable and resistant against exonuclease degradation than their linear counterparts. Therefore, circRNA can be a promising platform for RNA-based therapeutics. However, in the context of innate immunity, whether exogenous circRNA can evade immune surveillance remains to be fully elucidated. To explore this, a study was conducted in which circRNAs were synthesized via the permuted intron-exon (PIE) system with group I introns and transfected into immune cell models to determine its immunogenicity and the innate sensors involved in their recognition. FIG. 6 shows the mechanism of Group I intron-PIE splicing. Free guanosine attacks permuted linear template, leading to spontaneous circularization of the exonic sequences.

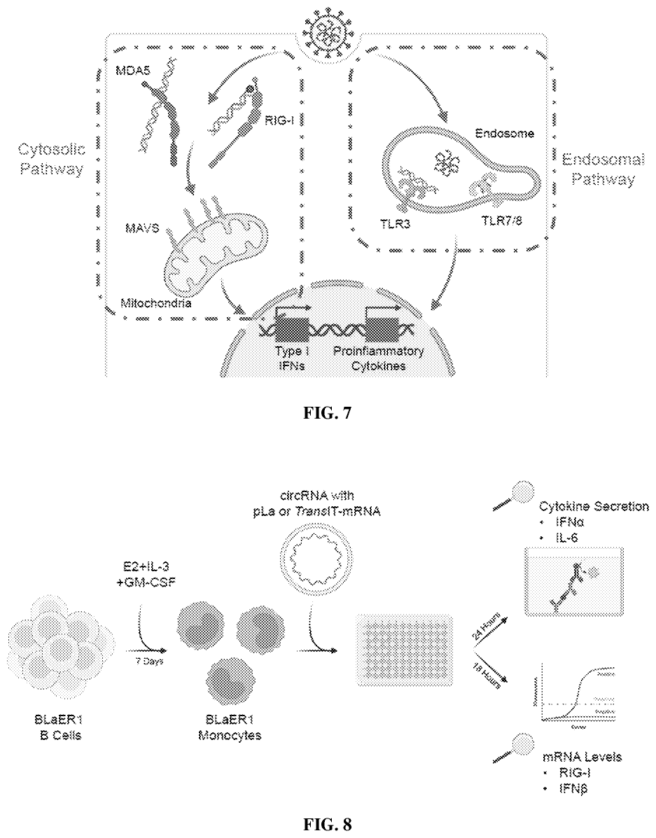

Central to this study is RNA sensing in innate immunity. FIG. 7 shows self vs. non-self-recognition protects host from invading pathogens. Detection of structures associated with foreign RNA by intracellular sensors results in a signaling cascade, ultimately inducing an innate immune response by transcriptional upregulation of Type I interferons (IFNs) and proinflammatory cytokines.

FIG. 8 shows a schematic of the experimental workflow used in this study. After BLaER1 cells were transdifferentiated from immune-insensitive pre-B cells to highly responsive monocytes/macrophages, they were transfected with circRNA complexed to either poly-L-arginine (pLa) or TransIT-mRNA. The pLa and TransIT-mRNA deliver RNA to the endolysosomal or cytoplasmic compartment, respectively. The study measured cytokine secretion via ELISA as a readout for immunogenicity. In addition, the study also determined the transcript induction of certain immune genes via RT-qPCR.

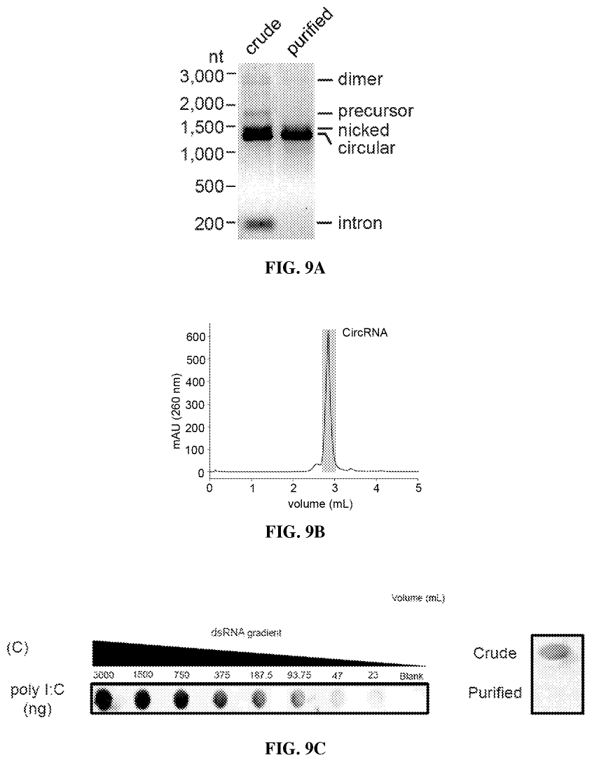

FIGS. 9A-9C shows circRNA preparation. circRNA was fully purified of any intermediate contaminants from splicing reactions. FIG. 9A shows the precursor IVT-mRNA and purified circRNA were analyzed by a glyoxal gel. FIG. 9B shows an FPLC chromatogram of purified circRNA. FIG. 9C shows 200 ng of RNA from the crude reaction and purified fraction were blotted and analyzed with J2 dsRNA-specific mAb, alongside a serially diluted positive control and blank.

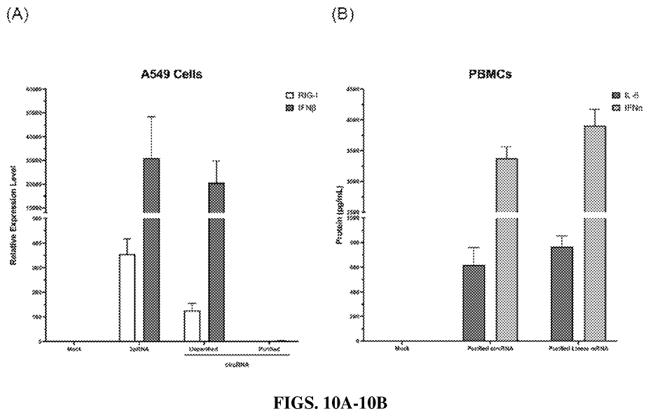

FIGS. 10A-10B show circRNA immunogenicity. Purified circRNA is non-immunogenic in non-immune cells but is immunogenic in primary immune cells. Following transfection of crude or purified fraction of circRNA, relative mRNA levels of RIG-I and IFNβ was determined in A549 cells (FIG. 10A), and cytokine release was profiled in PBMCs (FIG. 10B).

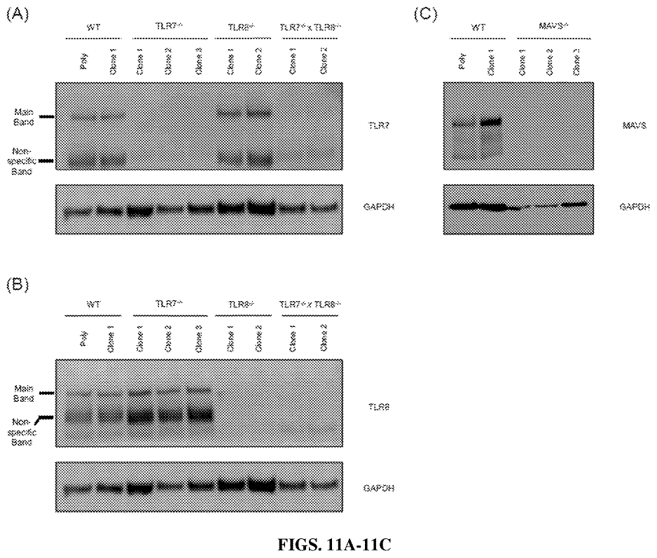

FIGS. 11A-11C show verification of CRISPR KO of gene-of-interest in transdifferentiated BLaER1 cells. TLR7 (FIG. 11A), TLR8 (FIG. 11B), or MAVS (FIG. 11C) expression of the indicated genotypes are shown.

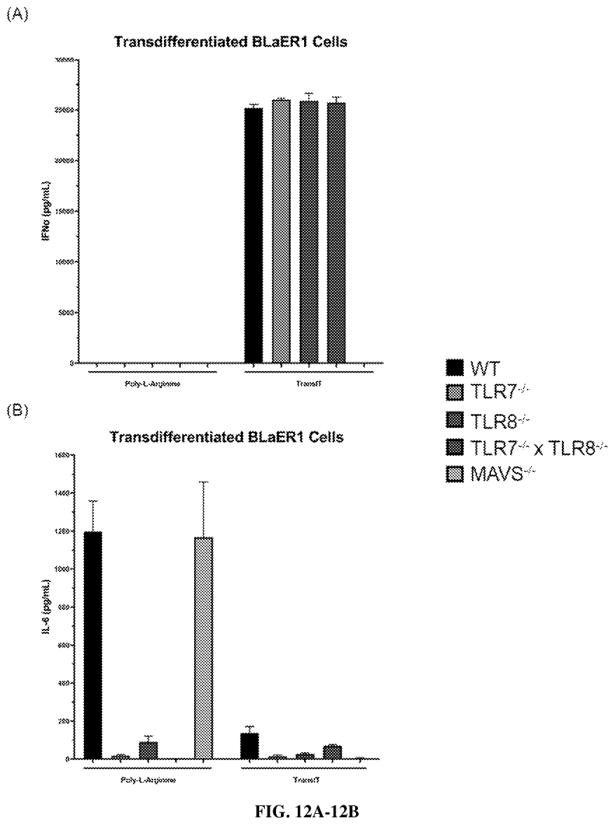

FIGS. 12A-12B show circRNA sensing is TLR7/8-mediated. The mode of circRNA delivery influences the ensuing cytokine/IFN responses in BLaER1 monocytes. FIG. 12A shows a MAVS-dependent Type I IFN response is induced when circRNA is introduced into the cytoplasm. FIG. 12B shows endosomal-delivered circRNA is sensed by TLR7/8 and activates the inflammatory program.

This study determined that purified circRNA do not activate non-immune cells (A549 cells) but are immunogenic in primary immune cells (PBMCs). Cytoplasmic detection of circRNA triggers a strong MAVS-dependent Type I IFN response, whereas endosomal delivery by pLa induces a potent TLR7/8-mediated proinflammatory response. However, pLa does not provoke detectable IFN signaling, and TransIT results in marginal inflammation. Future work can further investigate how circRNA activates cytosolic sensors via testing their immunogenicity in RIG-I−/− and MDA5−/− BLaER1 cell lines to precisely determine which of the two sensors is responsible. Future work can also assess whether immunogenicity persists across a variety of delivery methods (LNP, DOTAP, etc.), and which innate immune response they would induce: proinflammatory or antiviral. Similar to how nucleoside modifications of linear mRNA allows immune evasion, such work can also determine if the same effect can be seen in circRNA. Besides the PIE method, circRNA can be synthesized via splint ligation so that the constructs are “scarless,” and their immunogenicity can be tested in immune cell models.

### Example 3: ψ/m1ψ Modification Reduces the Activity of the Group I Intron

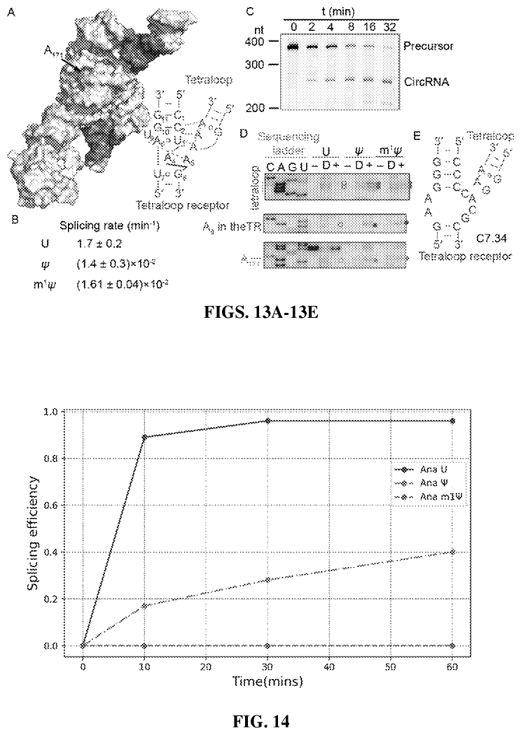

The RNA self-splicing group I intron has been a prominent model system for the study of RNA catalysis and structure-function relationships. Its catalytic core adopts an intricate fold and is further stabilized by contacts in the peripheral regions through GAAA tetraloop-tetraloop receptor (TR) interactions (FIG. 13A). The group I intron is also an important tool for circRNA therapeutics because a permuted Anabaena group I intron can circularize RNAs up to 5,000 nucleotides with an impressive 80% circularization efficiency (in contrast, the traditional enzymatic ligation method can only give<5% yield on such long RNAs). However, this Anabaena group I intron cannot circularize nucleoside-modified RNAs, because its activity is completely abolished by m1ψ modification. Therefore, engineering a group I intron compatible with modified nucleosides remains a key step for developing nucleoside-modified circRNAs.

FIGS. 13A-13E depict how w/m1ψ modification reduces group I intron activity by weakening the interaction between the GAAA tetraloop and its receptor. FIG. 13A shows the crystal structure of an Azoarcus group I intron and a schematic diagram of the structure of tetraloop receptor and its interactions with the GAAA tetraloop. FIG. 13B shows splicing rates of unmodified or ψ/m1ψ-modified Azoarcus group I introns. FIG. 13C shows efficient circularization of a 151-nt ψ-modified RNA. FIG. 13D shows DMS chemical probing of unmodified or ψ/m1ψ-modified Azoarcus group I introns. The absence and presence of DMS modification at key residues are highlighted by open and close circles, respectively. FIG. 13E shows the C7.34 tetraloop-tetraloop receptor pair discovered by in vitro selection does not contain U nucleotides.

A more compact group I intron has recently been identified from the thermophilic bacterium Azoarcus that tolerates ψ/m1ψ modifications, although its splicing rate was reduced by ~100-fold when modified (FIG. 13B). Using the Azoarcus group I intron, a 151-nt ψ-modified RNA was circularized at a >90% efficiency (FIG. 13C). This result established the plausibility of using the permuted group I intron strategy to prepare nucleoside-modified circRNAs. A deeper understanding of how ψ/m1ψ reduces the Azoarcus group I intron activity will allow further improvement of its splicing rate and circularization of long nucleoside-modified RNAs with high efficiency.

As a first step toward this goal, DMS chemical probing was used to examine the secondary structures of ψ/m1ψ-modified group I introns. While these modified group I introns retain the same overall secondary structures as the unmodified ones, significant changes were found in the tetraloop and its receptor. In the unmodified Azoarcus group I intron, strong DMS protection is observed in the first and second adenosines in the GAAA tetraloop and A8 in the TR, consistent with a stable tetraloop-TR interaction. However, ψ/m1ψ modifications rendered these adenosines reactive to DMS to the same extent as an unfolded RNA (FIG. 13D). Because the GAAA tetraloop does not contain any uridines, it is hypothesized that ψ/m1ψ alters the structure of the tetraloop receptor, significantly weakening its interaction with the GAAA tetraloop. Furthermore, ψ/m1ψ modification sensitizes nucleotide A171, which is buried deep in the catalytic core (FIG. 13A), to DMS modification (FIG. 13D), suggesting that the weakened tetraloop-tetraloop receptor interactions altered the structure of the catalytic core. Collectively, these results provide a clear mechanistic model of how ψ/m1ψ modification reduces the activity of the Azoarcus group I intron.

To further confirm that the structure of the TR is altered by these modified nucleotides, CMCT chemical probing can be performed to directly measure the base pairing of the U/w/m1ψ y, especially those in the TR. It is expected that U3, which base pairs with A8, will become reactive towards CMCT in modified group I introns. In addition, because 2′-OH groups are heavily involved in the tertiary interactions of the group I intron (particularly in the active site and the tetraloop-TR interactions), SHAPE experiments can also be performed as an orthogonal approach to study how these modified nucleotides alter the structure of the tetraloop and its receptor and affect the folding of the active site.

Building upon this mechanistic insight, mutations can be introduced to restore the GAAA tetraloop-TR interactions. Previous in vitro selection studies identified many sequences that can form strong tetraloop-TR interactions and fully support the splicing activity of the group I intron. Fortuitously, some of these sequences do not contain uridines (FIG. 13E), making them ideal candidates for this purpose. It is expected that introducing these new tetraloop/TR sequences can restore the activity of ψ/m1ψ-modified Azoarcus group I intron. If further optimization is required, well-established in vitro selection strategies can be used to find optimal sequences for circularizing ψ/m1ψ-modified RNAs. These experiments can allow engineering of a group I intron to circularize long nucleoside-modified RNAs.

### Example 4: Making Modified Circular RNA with the Azoarcus Intron

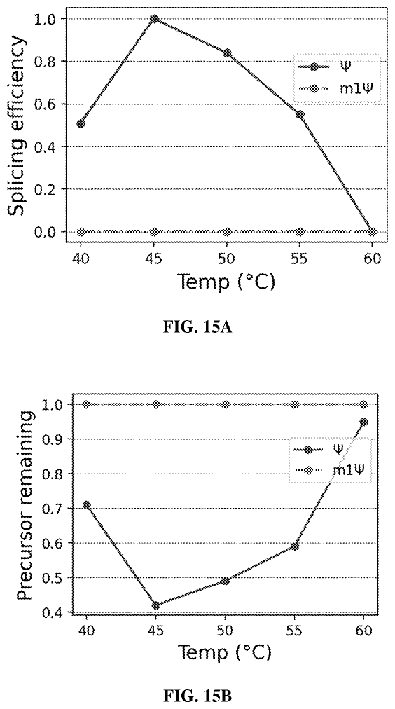

Generally, permuted intron-exon (PIE) circularization of RNA is inhibited by modified nucleotides such as N1-methylpseudouridine. Commonly, the Anabaena intron/exon is used for PIE circularization. FIG. 14 depicts the effects of pseudouridine and N1-methylpseudouridine on splicing efficiency of the Anabaena PIE, where it is observed that the modified uridine nucleotides considerably decrease splicing efficiency and RNA circularization. FIGS. 15A-15B depict the effects of pseudouridine and N1-methylpseudouridine on splicing efficiency of the Anabaena PIE and precursor remaining over time. A summary of the splicing rate is given in TABLE 1.

TABLE 1
Summary of splicing rate for Anabaena PIE.

| | | | m1Ψ (N1- |
|---|---|---|---|
| | U | Ψ (pseudouridine) | methylpseudouridine) |
| 50° C. | 2.2 +/− 0.3 | 0.017 +/− 0.001 | (not observed) |

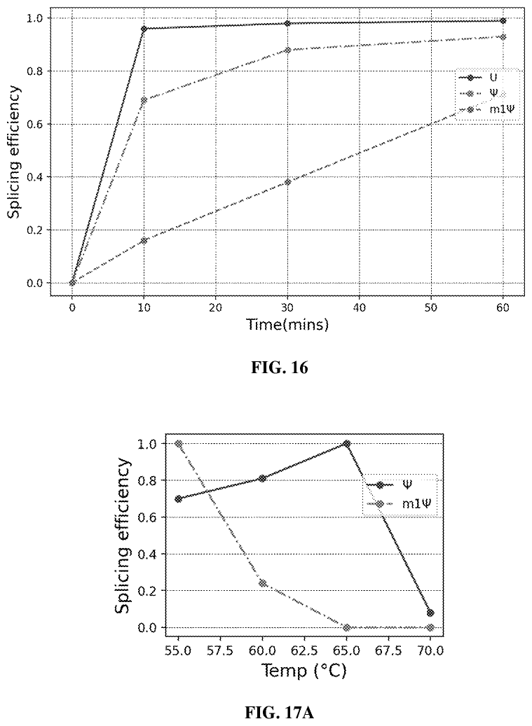

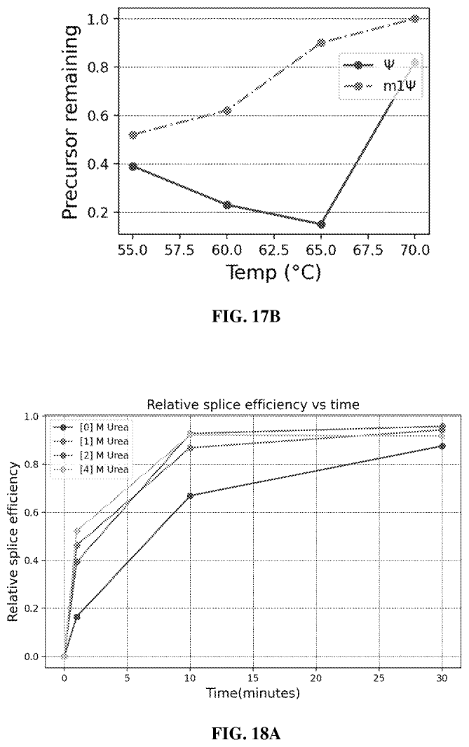

Surprisingly, the Azoarcus intron/exon is able to effectively circularize RNA even in the presence of modified uridine nucleotides. FIG. 16 depicts the effects of pseudouridine and N1-methylpseudouridine on splicing efficiency of the Azoarcus PIE, where it is observed that the Azoarcus PIE is still active even in the presence of modified uridine nucleotides. FIGS. 17A-17B depict the effects of pseudouridine and N1-methylpseudouridine on splicing efficiency of the Azoarcus PIE and precursor remaining over time. A summary of the splicing rate is given in TABLE 2.

TABLE 2
Summary of splicing rate for Azoarcus PIE.

| | | | m1Ψ (N1- |
|---|---|---|---|
| | U | Ψ (pseudouridine) | methylpseudouridine) |
| 50° C. | 1.7 +/− 0.2 | 0.014 +/− 0.003 | 0.0161 +/− 0.0004 |
| 60° C. | 2.8 +/− 0.1 | 0.29 +/− 0.06 | 0.028 +/− 0.002 |

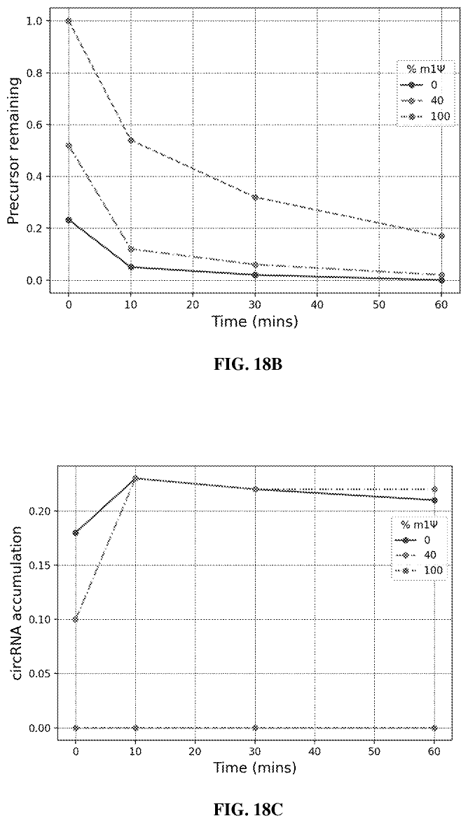

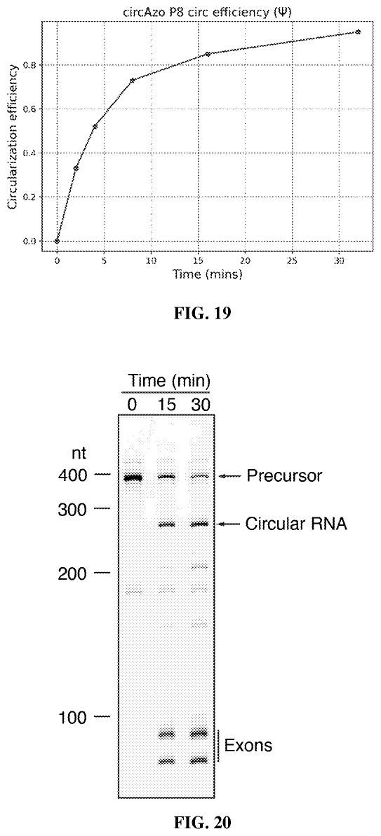

FIGS. 18A-18B depict that the Azoarcus PIE P6 is circularized efficiently and can successfully make modified circular mRNA. FIG. 18C depicts that the circular mRNA does not accumulate. FIG. 19 depicts the circularization efficiency of Azoarcus PIE P8.

### Example 5: Developing Nucleoside-Modified Circular mRNAs

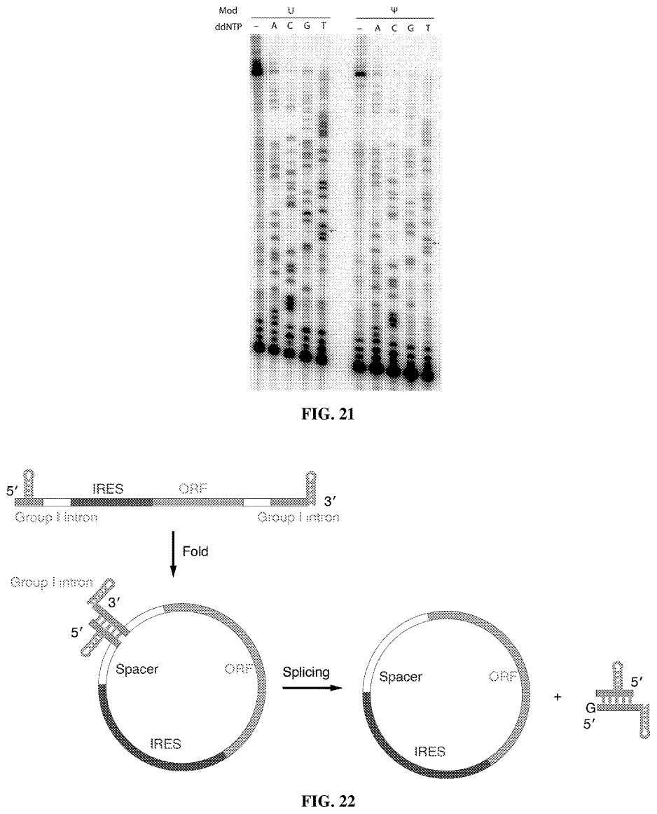

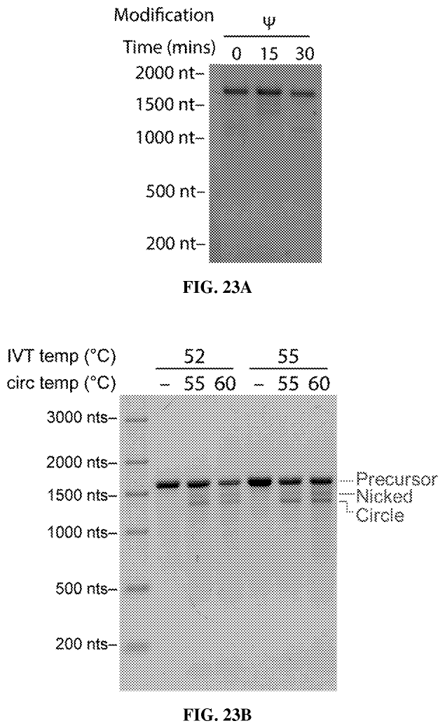

FIG. 20 shows the results of a 6% denaturing PAGE, SYBR gold stained assay which demonstrates the circularization of a Ψ-modified RNA (SEQ ID NO: 25) with greater than 90% efficiency. FIG. 21 depicts that the improved Azoarcus PIE generates a precise exon-exon junction. FIG. 22 is a schematic of the permuted intron-exon (PIE) strategy for circularization. FIGS. 23A-23B depict 15% circularization efficiency for a 1.5 kb pseudouridine-modified mRNA (SEQ ID NO: 26).

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y′”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g., desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the terms “optional” or “optionally” means that the subsequently described event or circumstance can or cannot occur, and that the description includes instances where said event or circumstance occurs and instances where it does not.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

Fusion polynucleotides also are contemplated herein. A “fusion polynucleotide” refers to the fusion of the nucleotide sequence of a first polynucleotide to the nucleotide sequence of a second heterologous polynucleotide (e.g., the 3′ end of a first polynucleotide to a 5′ end of the second polynucleotide). Where the first and second polynucleotides encode proteins, the fusion may be such that the encoded proteins are in-frame and results in a fusion protein. The first and second polynucleotide may be fused such that the first and second polynucleotide are operably linked (e.g., as a promoter and a gene expressed by the promoter as discussed below).

“Homology” refers to sequence similarity or, interchangeably, sequence identity, between two or more polypeptide sequences or polynucleotide sequences. Homology, sequence similarity, and percentage sequence identity may be determined using methods in the art and described herein.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide. A variant polynucleotide may have substantially the same functional activity as a reference polynucleotide.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 1 3, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

The term “mismatched” or “mismatched target sequence” refers to an off-target sequence that is not perfectly complementary to the first DNA sequence or the second DNA sequence of the chimeric deoxyribonucleic acid described herein. The dual retargeted DNA may have at least one mismatch, but can also have 2, 3, 4, 5, 6 or 7 or more mismatched nucleotides to the off-target sequence.

The following patents, applications and publications as listed below and throughout this document are hereby incorporated by reference in their entirety herein.

### REFERENCE LIST

1. Wesselhoeft, R. A. et al. Molecular Cell 74, 508-520.e4 (2019).
2. Strobel, S. A. & Shetty, K. PNAS 94, 2903-2908 (1997).
3. Zhao X, Zhong Y, Wang X, Shen J, An W. Int J Med Sci. 19(6):975-985. (2022)
4. Jensen S and Thomsen A R. J Virol. 86(6):2900-10. (2012)
5. Wesselhoeft R A, et al. Mol. Cell. 74(3):508-520.e4. (2019)
6. Chen Y G, et al. Mol Cell. 3; 76(1):96-109.e9. (2019)
7. Obi P and Chen Y G. Methods. 196:85-103. (2021)
8. Chen L L. Nat Rev Mol Cell Biol. (4):205-11. (2016)

SEQUENCES
SEQ ID NO: 1
GGGCCTCTAGCTCATGCTTGGTTAGAGCAGCGGACTCATATTTCGATGTGCCTTGCG
CCGGGAAACCACGCAAGGGATGGTGTCAAATTCGGCGAAACCTAAGCGCCCGCCCG
GGCGTATGGCAACGCCGAGCCAAGCTTCGGCGCCTGCGCCGATGAAGGTGTAGAGA
CTAGACGGCACCCACCTAAGGCAAACGCTATGGTGAAGGCATAGTCCAGGGAGTGG
CGAAAGTCACACAAACCGGAATCCGTTGGTGCTGGGTTCGACTCCCAGGGGGCCCA
CCA
SEQ ID NO: 2
GGCGCCGATGAAGGTGTAGAGACTAGACGGCACCCACCTAAGGCAAACGCTATGGT
GAAGGCATAGTCCAGGGAGTGGCGAAAGTCACACAAACCGG
SEQ ID NO: 3
GGGAGCTATGGTGAAGGCATAGTCCAGGGAGTGGCGAAAGTCACACAAACCGG
SEQ ID NO: 4
ATTTCGATGTGCCTTGCGCCGGGAAACCACGCAAGGGATGGTGTCAAATTCGGCGA
AACCTAAGCGCCCGCCCGGGCGTATGGCAACGCCGAGCCAAGCTTCGGCGCC
SEQ ID NO: 5
ATTTCGATGTGCCTTGCGCCGGGAAACCACGCAAGGGATGGTGTCAAATTCGGCGA
AACCTAAGCGCCCGCCCGGGCGTATGGCAACGCCGAGCCAAGCTTCGGCGCCTGCG
CCGATGAAGGTGTAGAGACTAGACGGCACCCACCTAAGGCTCAA
SEQ ID NO: 6
GGGCCTCTAGCTCATGCTTGGTTAGAGCAGCGGACTCAT
SEQ ID NO: 7
AATCCGTTGGTGCTGGGTTCGACTCCCAGGGGGCCCACCA
SEQ ID NO: 8
CGGATCCGAGACGATCTAGACTGCAGCGTCTCAGGGTCGAC
SEQ ID NO: 9
AAGGCGAGACGATAGGATCCCCACGTCTCCATCA
SEQ ID NO: 10
AAAAACAAAAACAAAAAAA
SEQ ID NO: 11
AAAAAACAAAAAACAAAAA
SEQ ID NO: 12
GGCGCCGATGAAGGTGTAGAGACTAGACGGCACCCACCTAAGGCAAACGCTATGGT
GAAGGCATAGTCCAGGGAGTGGCGAAAGTCACACAAACCGGAATCCGTTGGTGCTG
GGTTCGACTCCCAGGGGGCCCACCAAAAAACAAAAACAAAAAAACGGATCCGAGA
CGATCTAGACTGCAGCGTCTCAGGGTCGACAAAAAACAAAAAACAAAAAGGGCCTC
TAGCTCATGCTTGGTTAGAGCAGCGGACTCATATTTCGATGTGCCTTGCGCCGGGAA
ACCACGCAAGGGATGGTGTCAAATTCGGCGAAACCTAAGCGCCCGCCCGGGCGTAT
GGCAACGCCGAGCCAAGCTTCGGCGCC
SEQ ID NO: 13
GGGAGCTATGGTGAAGGCATAGTCCAGGGAGTGGCGAAAGTCACACAAACCGGAAT
CCGTTGGTGCTGGGTTCGACTCCCAGGGGGCCCACCAAAAAACAAAAACAAAAAAA
AAGGCGAGACGATAGGATCCCCACGTCTCCATCAAAAAAACAAAAAACAAAAAGG
GCCTCTAGCTCATGCTTGGTTAGAGCAGCGGACTCATATTTCGATGTGCCTTGCGCC
GGGAAACCACGCAAGGGATGGTGTCAAATTCGGCGAAACCTAAGCGCCCGCCCGGG
CGTATGGCAACGCCGAGCCAAGCTTCGGCGCCTGCGCCGATGAAGGTGTAGAGACT
AGACGGCACCCACCTAAGGCTCAA
SEQ ID NO: 14
AATCCGTTGGTGCTGGGTTCGACTCCCAGGGGGCCCACCAAAAAACAAAAACAAAA
AAAAAGGCGAGACGATAGGATCCCCACGTCTCCATCAAAAAAACAAAAAACAAAA
AGGGCCTCTAGCTCATGCTTGGTTAGAGCAGCGGACTCAT
SEQ ID NO: 15
GGGAGCUAUGGUGAAGGCAUAGUCCAGGGAGUGGCGAAAGUCACACAAACCGG
SEQ ID NO: 16
GGGCGCCGAUGAAGGUGUAGAGACUAGACGGCACCCACCUAAGGCAAACGCUAUG
GUGAAGGCAUAGUCCAGGGAGUGGCGAAAGUCACACAAACCGG
SEQ ID NO: 17
AUUUCGAUGUGCCUUGCGCCGGGAAACCACGCAAGGGAUGGUGUCAAAUUCGGC
GAAACCUAAGCGCCCGCCCGGGCGUAUGGCAACGCCGAGCCAAGCUUCGGCGCCU
GCGCCGAUGAAGGUGUAGAGACUAGACGGCACCCACCUAAGGCUCAA
SEQ ID NO: 18
AUUUCGAUGUGCCUUGCGCCGGGAAACCACGCAAGGGAUGGUGUCAAAUUCGGC
GAAACCUAAGCGCCCGCCCGGGCGUAUGGCAACGCCGAGCCAAGCUUCGGCGCC
SEQ ID NO: 19
AAUCCGUUGGUGCUGGGUUCGACUCCCAGGGGGCCCACCA
SEQ ID NO: 20
AAUCCGUUGGUGCUGGGUUCGACUCCCAGGGGGCCCACCACGCCGGAAACGCAAU
AGCCG
SEQ ID NO: 21
GGGCCUCUAGCUCAUGCUUGGUUAGAGCAGCGGACUCAU
SEQ ID NO: 22
CGGCUAUUAUGCGUUACCGGCGGGGCCUCUAGCUCAUGCUUGGUUAGAGCAGCGG
ACUCAU
SEQ ID NO: 23
ACGUUACUGGCCGAAGCCGCUUGGAAUAAGGCCGGUGUGCGUUUGUCUAUAUGU
UAUUUUCCACCAUAUUGCCGUCUUUUGGCAAUGUGAGGGCCCGGAAACCUGGCCC
UGUCUUCUUGACGAGCAUUCCUAGGGGUCUUUCCCCUCUCGCCAAAGGAAUGCAA
GGUCUGUUGAAUGUCGUGAAGGAAGCAGUUCCUCUGGAAGCUUCUUGAAGACAA
ACAACGUCUGUAGCGACCCUUUGCAGGCAGCGGAACCCCCCACCUGGCGACAGGU
GCCUCUGCGGCCAAAAGCCACGUGUAUAAGAUACACCUGCAAAGGCGGCACAACC
CCAGUGCCACGUUGUGAGUUGGAUAGUUGUGGAAAGAGUCAAAUGGCUCUCCUC
AAGCGUAUUCAACAAGGGGCUGAAGGAUGCCCAGAAGGUACCCCAUUGUAUGGG
AUCUGAUCUGGGGCCUCGGUGCACAUGCUUUACAUGUGUUUAGUCGAGGUUAAA
AAACGUCUAGGCCCCCCGAACCACGGGGACGUGGUUUUCCUUUGAAAAACACGAU
GAUAAU
SEQ ID NO: 24
AUGGUGAGCAAGGGCGAGGAGCUGUUCACCGGGGUGGUGCCCAUCCUGGUCGAG
CUGGACGGCGACGUAAACGGCCACAAGUUCAGCGUGUCCGGCGAGGGCGAGGGCG
AUGCCACCUACGGCAAGCUGACCCUGAAGUUCAUCUGCACCACCGGCAAGCUGCC
CGUGCCCUGGCCCACCCUCGUGACCACCCUGACCUACGGCGUGCAGUGCUUCAGC
CGCUACCCCGACCACAUGAAGCAGCACGACUUCUUCAAGUCCGCCAUGCCCGAAG
GCUACGUCCAGGAGCGCACCAUCUUCUUCAAGGACGACGGCAACUACAAGACCCG
CGCCGAGGUGAAGUUCGAGGGCGACACCCUGGUGAACCGCAUCGAGCUGAAGGGC
AUCGACUUCAAGGAGGACGGCAACAUCCUGGGGCACAAGCUGGAGUACAACUACA
ACAGCCACAACGUCUAUAUCAUGGCCGACAAGCAGAAGAACGGCAUCAAGGUGAA
CUUCAAGAUCCGCCACAACAUCGAGGACGGCAGCGUGCAGCUCGCCGACCACUAC
CAGCAGAACACCCCCAUCGGCGACGGCCCCGUGCUGCUGCCCGACAACCACUACC
UGAGCACCCAGUCCGCCCUGAGCAAAGACCCCAACGAGAAGCGCGAUCACAUGGU
CCUGCUGGAGUUCGUGACCGCCGCCGGGAUCACUCUCGGCAUGGACGAGCUGUAC
AAGUAA
SEQ ID NO: 25
GGGAGCUAUGGUGAAGGCAUAGUCCAGGGAGUGGCGAAAGUCACACAAACCGGA
AUCCGUUGGUGCUGGGUUCGACUCCCAGGGGGCCCACCAAAAAACAAAAACAAAA
AAAAAGGCGAGACGAUAGGAUCCCCACGUCUCCAUCAAAAAAACAAAAAACAAA
AAGGGCCUCUAGCUCAUGCUUGGUUAGAGCAGCGGACUCAUAUUUCGAUGUGCC
UUGCGCCGGGAAACCACGCAAGGGAUGGUGUCAAAUUCGGCGAAACCUAAGCGCC
CGCCCGGGCGUAUGGCAACGCCGAGCCAAGCUUCGGCGCCUGCGCCGAUGAAGGU
GUAGAGACUAGACGGCACCCACCUAAGGCUCAA
SEQ ID NO: 26
GGGCGCCGAUGAAGGUGUAGAGACUAGACGGCACCCACCUAAGGCAAACGCUAUG
GUGAAGGCAUAGUCCAGGGAGUGGCGAAAGUCACACAAACCGGAAUCCGUUGGU
GCUGGGUUCGACUCCCAGGGGGCCCACCACGCCGGAAACGCAAUAGCCGAAAAAC
AAAAACAAAAAAAAAGGCGAGACGACGUUACUGGCCGAAGCCGCUUGGAAUAAG
GCCGGUGUGCGUUUGUCUAUAUGUUAUUUUCCACCAUAUUGCCGUCUUUUGGCA
AUGUGAGGGCCCGGAAACCUGGCCCUGUCUUCUUGACGAGCAUUCCUAGGGGUCU
UUCCCCUCUCGCCAAAGGAAUGCAAGGUCUGUUGAAUGUCGUGAAGGAAGCAGU
UCCUCUGGAAGCUUCUUGAAGACAAACAACGUCUGUAGCGACCCUUUGCAGGCAG
CGGAACCCCCCACCUGGCGACAGGUGCCUCUGCGGCCAAAAGCCACGUGUAUAAG
AUACACCUGCAAAGGCGGCACAACCCCAGUGCCACGUUGUGAGUUGGAUAGUUGU
GGAAAGAGUCAAAUGGCUCUCCUCAAGCGUAUUCAACAAGGGGCUGAAGGAUGC
CCAGAAGGUACCCCAUUGUAUGGGAUCUGAUCUGGGGCCUCGGUGCACAUGCUUU
ACAUGUGUUUAGUCGAGGUUAAAAAACGUCUAGGCCCCCCGAACCACGGGGACGU
GGUUUUCCUUUGAAAAACACGAUGAUAAUAUGGUGAGCAAGGGCGAGGAGCUGU
UCACCGGGGUGGUGCCCAUCCUGGUCGAGCUGGACGGCGACGUAAACGGCCACAA
GUUCAGCGUGUCCGGCGAGGGCGAGGGCGAUGCCACCUACGGCAAGCUGACCCUG
AAGUUCAUCUGCACCACCGGCAAGCUGCCCGUGCCCUGGCCCACCCUCGUGACCA
CCCUGACCUACGGCGUGCAGUGCUUCAGCCGCUACCCCGACCACAUGAAGCAGCA
CGACUUCUUCAAGUCCGCCAUGCCCGAAGGCUACGUCCAGGAGCGCACCAUCUUC
UUCAAGGACGACGGCAACUACAAGACCCGCGCCGAGGUGAAGUUCGAGGGCGACA
CCCUGGUGAACCGCAUCGAGCUGAAGGGCAUCGACUUCAAGGAGGACGGCAACAU
CCUGGGGCACAAGCUGGAGUACAACUACAACAGCCACAACGUCUAUAUCAUGGCC
GACAAGCAGAAGAACGGCAUCAAGGUGAACUUCAAGAUCCGCCACAACAUCGAGG
ACGGCAGCGUGCAGCUCGCCGACCACUACCAGCAGAACACCCCCAUCGGCGACGG
CCCCGUGCUGCUGCCCGACAACCACUACCUGAGCACCCAGUCCGCCCUGAGCAAA
GACCCCAACGAGAAGCGCGAUCACAUGGUCCUGCUGGAGUUCGUGACCGCCGCCG
GGAUCACUCUCGGCAUGGACGAGCUGUACAAGUAACGUCUCAAUCAAAAAAACA
AAAAACAAAAACGGCUAUUAUGCGUUACCGGCGGGGCCUCUAGCUCAUGCUUGG
UUAGAGCAGCGGACUCAUAUUUCGAUGUGCCUUGCGCCGGGAAACCACGCAAGGG
AUGGUGUCAAAUUCGGCGAAACCUAAGCGCCCGCCCGGGCGUAUGGCAACGCCGA
GCCAAGCUUCGGCGCC

1. An RNA vector comprising:
a permuted group 1 intron/exon comprising a 5′ exon, a 3′ exon, and an intron, the intron comprising a 5′ intron end and a 3′ intron end;
at least one modified nucleotide selected from pseudouridine or N1-methylpseudouridine; and
a nucleic acid sequence encoding a target protein;
wherein the 3′ exon is upstream of the 5′ exon;
wherein a portion of the intron including the 3′ intron end is upstream of the 3′ exon;
wherein a portion of the intron including the 5′ intron end is downstream of the 5′ exon; and
wherein the permuted group 1 intron/exon is active in the presence of the modified nucleotide.

2-3. (canceled)

4. The RNA vector of claim 1, wherein the permuted group 1 intron/exon further comprises at least one mutation to a wildtype Azoarcus group 1 intron/exon.

5-6. (canceled)

7. The RNA vector of claim 1, wherein the portion of the intron including the 3′ intron end comprises 80% similarity or more to SEQ ID NO: 15 or SEQ ID NO: 16; and
wherein the portion of the intron including the 5′ intron end comprises 80% similarity or more to SEQ ID NO: 17 or SEQ ID NO: 18.

8. (canceled)

9. The RNA vector of claim 1, wherein the 3′ exon comprises 80% similarity or more to SEQ ID NO: 19 or SEQ ID NO: 20; and
wherein the 5′ exon comprises 80% similarity or more to SEQ ID NO: 21 or SEQ ID NO: 22.

10. (canceled)

11. The RNA vector of claim 1, further comprising an internal ribosome entry site (IRES) comprising 80% similarity or more to SEQ ID NO: 23.

12-13. (canceled)

14. The RNA vector of claim 1, further comprising at least one spacer.

15. The RNA vector of claim 14, wherein the spacer is selected at each occurrence from a sequence comprising 80% similarity or more to SEQ ID NO: 10 or SEQ ID NO: 11.

16. The RNA vector of claim 1, wherein the vector is greater than about 1000 nucleotides in length.

17. A composition comprising a circular RNA comprising an internal ribosome entry site (IRES) and a nucleic acid sequence encoding a therapeutic protein greater than 200 amino acids in length, further comprising at least one modified nucleotide selected from pseudouridine or N1-methylpseudouridine vector.

18. (canceled)

19. A DNA vector comprising:
a permuted group 1 intron/exon comprising a 5′ exon, a 3′ exon, and an intron, the intron comprising a 5′ intron end and a 3′ intron end; and
a multiple cloning site;
wherein the 3′ exon is upstream of the 5′ exon;
wherein a portion of the intron including the 3′ intron end is upstream of the 3′ exon; and
wherein a portion of the intron including the 5′ intron end is downstream of the 5′ exon.

20-34. (canceled)


SEQUENCE LISTING (26 sequences)

SEQ ID NO: 1 - DNA, 284 bp, synthetic construct
  misc_feature 1..284: note = Description of sequence: vector component
  source 1..284: mol_type = other DNA
gggcctctagctcatgcttggttagagcagcggactcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggcaaacgctatggtgaaggcatagtccagggagtggcgaaagtcacacaaaccggaatccgttggtgctgggttcgactcccagggggcccacca

SEQ ID NO: 2 - DNA, 97 bp, synthetic construct
  misc_feature 1..97: note = Description of sequence: vector component
  source 1..97: mol_type = other DNA
ggcgccgatgaaggtgtagagactagacggcacccacctaaggcaaacgctatggtgaaggcatagtccagggagtggcgaaagtcacacaaaccgg

SEQ ID NO: 3 - DNA, 53 bp, synthetic construct
  misc_feature 1..53: note = Description of sequence: vector component
  source 1..53: mol_type = other DNA
gggagctatggtgaaggcatagtccagggagtggcgaaagtcacacaaaccgg

SEQ ID NO: 4 - DNA, 108 bp, synthetic construct
  misc_feature 1..108: note = Description of sequence: vector component
  source 1..108: mol_type = other DNA
atttcgatgtgccttgcgccgggaaaccacgcaagggatggtgtcaaattcggcgaaacctaagcgcccgcccgggcgtatggcaacgccgagccaagcttcggcgcc

SEQ ID NO: 5 - DNA, 156 bp, synthetic construct
  misc_feature 1..156: note = Description of sequence: vector component
  source 1..156: mol_type = other DNA
atttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggctcaa

SEQ ID NO: 6 - DNA, 39 bp, synthetic construct
  misc_feature 1..39: note = Description of sequence: vector component
  source 1..39: mol_type = other DNA
gggcctctagctcatgcttggttagagcagcggactcat

SEQ ID NO: 7 - DNA, 40 bp, synthetic construct
  misc_feature 1..40: note = Description of sequence: vector component
  source 1..40: mol_type = other DNA
aatccgttggtgctgggttcgactcccagggggcccacca

SEQ ID NO: 8 - DNA, 41 bp, synthetic construct
  misc_feature 1..41: note = Description of sequence: vector component
  source 1..41: mol_type = other DNA
cggatccgagacgatctagactgcagcgtctcagggtcgac

SEQ ID NO: 9 - DNA, 34 bp, synthetic construct
  misc_feature 1..34: note = Description of sequence: vector component
  source 1..34: mol_type = other DNA
aaggcgagacgataggatccccacgtctccatca

SEQ ID NO: 10 - DNA, 19 bp, synthetic construct
  misc_feature 1..19: note = Description of sequence: vector component
  source 1..19: mol_type = other DNA
aaaaacaaaaacaaaaaaa

SEQ ID NO: 11 - DNA, 19 bp, synthetic construct
  misc_feature 1..19: note = Description of sequence: vector component
  source 1..19: mol_type = other DNA
aaaaaacaaaaaacaaaaa

SEQ ID NO: 12 - DNA, 363 bp, synthetic construct
  misc_feature 1..363: note = Description of sequence: vector component
  source 1..363: mol_type = other DNA
ggcgccgatgaaggtgtagagactagacggcacccacctaaggcaaacgctatggtgaaggcatagtccagggagtggcgaaagtcacacaaaccggaatccgttggtgctgggttcgactcccagggggcccaccaaaaaacaaaaacaaaaaaacggatccgagacgatctagactgcagcgtctcagggtcgacaaaaaacaaaaaacaaaaagggcctctagctcatgcttggttagagcagcggactcatatttcgatgtgccttgcgccgggaaaccacgcaagggatggtgtcaaattcggcgaaacctaagcgcccgcccgggcgtatggcaacgccgagccaagcttcggcgcc

SEQ ID NO: 13 - DNA, 360 bp, synthetic construct
  misc_feature 1..360: note = Description of sequence: vector component
  source 1..360: mol_type = other DNA
gggagctatggtgaaggcatagtccagggagtggcgaaagtcacacaaaccggaatccgttggtgctgggttcgactcccagggggcccaccaaaaaacaaaaacaaaaaaaaaggcgagacgataggatccccacgtctccatcaaaaaaacaaaaaacaaaaagggcctctagctcatgcttggttagagcagcggactcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggctcaa

SEQ ID NO: 14 - DNA, 151 bp, synthetic construct
  misc_feature 1..151: note = Description of sequence: vector component
  source 1..151: mol_type = other DNA
aatccgttggtgctgggttcgactcccagggggcccaccaaaaaacaaaaacaaaaaaaaaggcgagacgataggatccccacgtctccatcaaaaaaacaaaaaacaaaaagggcctctagctcatgcttggttagagcagcggactcat

SEQ ID NO: 15 - RNA, 53 nt, synthetic construct
  misc_feature 1..53: note = Description of sequence: vector component
  source 1..53: mol_type = other RNA
gggagctatggtgaaggcatagtccagggagtggcgaaagtcacacaaaccgg

SEQ ID NO: 16 - RNA, 98 nt, synthetic construct
  misc_feature 1..98: note = Description of sequence: vector component
  source 1..98: mol_type = other RNA
gggcgccgatgaaggtgtagagactagacggcacccacctaaggcaaacgctatggtgaaggcatagtccagggagtggcgaaagtcacacaaaccgg

SEQ ID NO: 17 - RNA, 156 nt, synthetic construct
  misc_feature 1..156: note = Description of sequence: vector component
  source 1..156: mol_type = other RNA
atttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggctcaa

SEQ ID NO: 18 - RNA, 108 nt, synthetic construct
  misc_feature 1..108: note = Description of sequence: vector component
  source 1..108: mol_type = other RNA
atttcgatgtgccttgcgccgggaaaccacgcaagggatggtgtcaaattcggcgaaacctaagcgcccgcccgggcgtatggcaacgccgagccaagcttcggcgcc

SEQ ID NO: 19 - RNA, 40 nt, synthetic construct
  misc_feature 1..40: note = Description of sequence: vector component
  source 1..40: mol_type = other RNA
aatccgttggtgctgggttcgactcccagggggcccacca

SEQ ID NO: 20 - RNA, 60 nt, synthetic construct
  misc_feature 1..60: note = Description of sequence: vector component
  source 1..60: mol_type = other RNA
aatccgttggtgctgggttcgactcccagggggcccaccacgccggaaacgcaatagccg

SEQ ID NO: 21 - RNA, 39 nt, synthetic construct
  misc_feature 1..39: note = Description of sequence: vector component
  source 1..39: mol_type = other RNA
gggcctctagctcatgcttggttagagcagcggactcat

SEQ ID NO: 22 - RNA, 61 nt, synthetic construct
  misc_feature 1..61: note = Description of sequence: vector component
  source 1..61: mol_type = other RNA
cggctattatgcgttaccggcggggcctctagctcatgcttggttagagcagcggactcat

SEQ ID NO: 23 - RNA, 551 nt, synthetic construct
  misc_feature 1..551: note = Description of sequence: vector component
  source 1..551: mol_type = other RNA
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 24 - RNA, 720 nt, synthetic construct
  misc_feature 1..720: note = Description of sequence: vector component
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 25 - RNA, 360 nt, synthetic construct
  misc_feature 1..360: note = Description of sequence: vector component
  source 1..360: mol_type = other RNA
gggagctatggtgaaggcatagtccagggagtggcgaaagtcacacaaaccggaatccgttggtgctgggttcgactcccagggggcccaccaaaaaacaaaaacaaaaaaaaaggcgagacgataggatccccacgtctccatcaaaaaaacaaaaaacaaaaagggcctctagctcatgcttggttagagcagcggactcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggctcaa

SEQ ID NO: 26 - RNA, 1658 nt, synthetic construct
  misc_feature 1..1658: note = Description of sequence: vector component
  source 1..1658: mol_type = other RNA
gggcgccgatgaaggtgtagagactagacggcacccacctaaggcaaacgctatggtgaaggcatagtccagggagtggcgaaagtcacacaaaccggaatccgttggtgctgggttcgactcccagggggcccaccacgccggaaacgcaatagccgaaaaacaaaaacaaaaaaaaaggcgagacgacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgtctcaatcaaaaaaacaaaaaacaaaaacggctattatgcgttaccggcggggcctctagctcatgcttggttagagcagcggactcatatttcgatgtgccttgcgccgggaaaccacgcaagggatggtgtcaaattcggcgaaacctaagcgcccgcccgggcgtatggcaacgccgagccaagcttcggcgcc